(19) United States
(12) Patent Application Publication
(10) Pub. No.: US 2026/0194520 A1
(43) Pub. Date: Jul. 9, 2026

(54) IN VITRO DETECTION METHOD AND DEVICE

(71) Applicant: ELFSCIENCE AB, Uppsala (SE)

(72) Inventor: Johan ELF, Uppsala (SE)

(21) Appl. No.: 19/132,649

(22) PCT Filed: Sep. 11, 2023
(86) PCT No.: PCT/SE2023/050890

§ 371 Date: May 23, 2025

(30) Foreign Application Priority Data
Nov. 29, 2022 (SE) 2251386-5

(51) Int. Cl. G01N 33/543; G01N 21/78

(52) U.S. Cl. CPC G01N 33/54388; G01N 21/78

(57) ABSTRACT

An in vitro detection method comprises contacting, for each detection channel (10, 20, 30, 40) of N≥2 detection channels (10, 20, 30, 40), a sample with a plurality of recognition molecules. Each recognition molecule binds specifically to or reacts specifically with a respective subset of at least one analyte. The plurality of recognition molecules are divided into multiple detection sets comprising multiple recognition molecules. The multiple recognition molecules of each detection set produce a same readout signal when binding specifically to or reacting specifically with an analyte. The N detection channels (10, 20, 30, 40) each have different detection sets or different combinations of detection sets. The method also comprises detecting, for each detection channel (10, 20, 30, 40), a readout signal (15, 25, 35, 45) produced by a recognition molecule binding specifically to or reacting specifically with an analyte. The method further comprises identifying an analyte in the sample based on a barcode representing the readout signals (15, 25, 35, 45) produced for the N detection channels (10, 20, 30, 40).

### TECHNICAL FIELD

The present invention generally relates to an in vitro detection method and device, and in particular to such a method and device capable of detecting an analyte out of a plurality of possible analytes in sample.

### BACKGROUND

A common in vitro diagnostics test for detecting an analyte or target molecule in a sample is the lateral flow test (LFT), also referred as lateral flow device (LFD) or rapid test in the art. When the analyte is a biological antigen, the recognition elements of the LFT are often specific antibodies and the LTF relies on immunoassay principles giving rise to the so-called lateral flow immunoassay (LFIA) or lateral flow immunochromatographic assay (LFIA). LFTs are simple devices intended to detect the presence of an analyte in a liquid sample without the need for specialized and costly equipment. LFTs are widely used in medical diagnostics in the home, at the point of care, and in the laboratory. For instance, the home pregnancy test is an LFT that detects a specific hormone.

LFTs are usually developed for the detection of a single analyte per assay. However, there is a need for simultaneous on-site measurement of different analytes from a single sample. The capability of such a multiplexing has several benefits, including improving the efficiency of testing and reducing costs, and is strongly requested for applications, in which advanced decision-making is requested or availability of samples is limited. Multiplexing strategies for LFTs can generally be divided into involving modification of the architecture of the device and those that are based on the use of specific probes.

The architecture-modifying multiplexing strategy typically involves providing an array of individual LFT strips arranged in a special holder designed to collect one sample and distribute it between the individual LFT strips. Each strip is then a traditional LFT strip that detects a single analyte. The number of analytes that can be detected by such an array thereby equals the number of individual LFT strips in the array. Another strategy involves spatial separation of detection sites on a single LFT strip. In such a case, several test lines or dots are designed on the LFT strip. There are, though, limitations with this strategy including reproducibility of the properties of the nitrocellulose membrane in the LFT strip, flow requirements, amount of sample applied to the LFT strip, robustness due to flow inhomogeneity, among others. As a consequence, there is an upper limit to the number of different analytes that can be detected with this multiplexing strategy.

Specific probes can be used in multiplexing strategies to detect different analytes in a sample on a single LFT strip. For instance, class-selective antibodies could be used as broad-specific recognition elements capable of detecting several analytes in a class. Alternatively, various labels, such as enzymes, fluorophores and nanoparticles, could be used to distinguish different analytes in a single LFT strip.

Anfossi et al., Multiplex lateral flow immunoassay: An overview of strategies towards high-throughput point-of-need testing, Biosensor 2019, 9:2 and Huang et al., Multiplexed detection of biomarkers in lateral-flow immunoassays, Analyst 2020, 145:2828-2840 provide overviews of strategies for multiplexing LFIAs.

There is still a need for an in vitro detection method and device that can be used to detect presence of an analyte out of a plurality of possible analytes in a sample.

### SUMMARY

It is a general objective to provide an in vitro detection method and device that can be used to detect presence of an analyte out of a plurality of possible analytes in a sample.

This and other objectives are met by embodiments as disclosed herein.

The present invention is defined in the independent claims. Further embodiment of the invention are defined in the dependent claims.

An aspect of the invention relates to an in vitro detection method comprising contacting, for each detection channel of N≥2 detection channels, a sample with a plurality of recognition molecules. Each recognition molecule of the plurality of recognition molecules binds specifically to or reacts specifically with a respective subset of at least one analyte. The plurality of recognition molecules are divided into multiple detection sets comprising multiple recognition molecules of the plurality of recognition molecules. The multiple recognition molecules of each detection set of the multiple detection sets produce a same readout signal when binding specifically to or reacting specifically with an analyte. The N detection channels each have different detection sets or different combinations of detection sets. The method also comprises detecting, for each detection channel of the N detection channels, any readout signal produced by a recognition molecule binding specifically to or reacting specifically with an analyte. The method further comprises identifying an analyte in the sample based on a barcode representing the readout signals produced for the N detection channels.

Another aspect of the invention relates to an in vitro detection device comprising N≥2 detection channels and a plurality of recognition molecules. Each recognition molecule of the plurality of recognition molecules binds specifically to or reacts specifically with a respective subset of at least one analyte. The plurality of recognition molecules are divided into multiple detection sets comprising multiple recognition molecules of the plurality of recognition molecules. The multiple recognition molecules of each detection set of the multiple detection sets produce a same readout signal when binding specifically to or reacting specifically with an analyte. The N detection channels each have different detection sets or different combinations of detection sets. Each analyte out of a plurality of possible analytes is assigned a unique barcode representing readout signals produced for the N detection channels. Each readout signal of the N readout signals is produced by a recognition molecule binding specifically to or reacting specifically with an analyte for each detection channel of the N detection channels.

The present invention solves the problem of detecting the presence of one or more analytes in a sample using fewer detection channels than the number of potential analytes. According to the invention, a combinatorial multiplexing of the readout signals from the detection channels produces a barcode that enables identification of any analyte in the sample.

### BRIEF DESCRIPTION OF THE DRAWINGS

The embodiments, together with further objects and advantages thereof, may best be understood by making reference to the following description taken together with the accompanying drawings, in which:

FIG. 1 is a schematic illustration of a portion of an in vitro detection device according to an embodiment;

FIG. 2 is a schematic illustration of a portion of an in vitro detection device according to another embodiment;

FIG. 3 is a schematic illustration of a portion of an in vitro detection device according to a further embodiment;

FIG. 4 is a schematic illustration of a portion of an in vitro detection device according to yet another embodiment;

FIG. 5 is a schematic illustration of readout signals from in vitro detection devices according to an embodiment;

FIG. 6 is a schematic illustration of readout signals from in vitro detection device according to another embodiment;

FIG. 7 is a schematic illustration of a substrate comprising multiple detection channels according to an embodiment;

FIG. 8 is a flow chart illustrating an in vitro detection method according to an embodiment; and

FIG. 9 is schematic illustration of an in vitro detection device according to an embodiment.

### DETAILED DESCRIPTION

The present invention generally relates to an in vitro detection method and device, and in particular to such a method and device capable of detecting an analyte out of a plurality of possible analytes in sample.

LFTs and similar in vitro detection tests and devices are generally useful when determining the presence or absence of a single analyte in a sample. However, when the number of potential analytes that may be present in the sample increases, the LFTs suffer from complexity in the design since they either need one LFT per analyte or merely a limited number of different analytes can be detected in a single LFT using a multiplexing strategy. Similarly, a genetic test based on, for example PCR, typically tests for one mutation or gene per test, although there are typically may different mutations that you want to screen for at the same time.

The present invention utilizes a combinatorial multiplexed readout for in vitro detection of analytes. This means that the presence of an analyte in a sample out of a plurality of possible analytes can be detected by combining readout signals from multiple detection channels into a single barcode. Hence, the readout signal from a single detection channel is not sufficient to unambiguously identify a given analyte. However, by combining the readout signals from the multiple detection channels into a barcode the given analyte can be identified. This combinatorial multiplexing of readout signals from multiple detection channels means that the presence of an analyte out of many potential analytes in a sample can be detected using fewer detection channels than the number of potential analytes. As an illustrative, but non-limiting, example, up to C^{N} different analytes can be identified using merely N detection channels with C different readout signals per channel. For example, readout signals in the form of three different colors would enable identification of up to 27 different analytes using merely three different detection channels.

As used herein, “analyte”, also referred to as “target molecule” herein, is any substance, molecule or chemical or biological constituent that can be detected in the in vitro detection method. In particular, the analyte could be a macromolecule, such as a protein, polypeptide or a nucleic acid molecule, such as a deoxyribonucleic acid (DNA) molecule, a ribonucleic acid (RNA) molecule, or a complementary DNA (cDNA) molecule. Hence, in a particular embodiment, the analyte is selected from the group consisting of a protein, a metabolite, a polypeptide and a nucleic acid molecule, preferably selected from the group consisting of a protein, a polypeptide and a nucleic acid molecule.

The “sample” could be any sample comprising one or multiple analytes to detect. The sample could, for instance, be a biological sample, such a body fluid sample, including a processed body fluid sample. Illustrative, but non-limiting, examples of body fluid samples include urine, blood, plasma, serum, amniotic fluid, cerebrospinal fluid, lymph, saliva and synovial fluid. A processed body fluid sample as used herein is a body fluid sample that has been processed in some way. For example, a body fluid sample could be filtered or separated into different sample portions to get a processed body fluid sample, such as a serum or plasma sample obtained from blood. Another example of a biological sample is a solid body sample, such as a biopsy, in which cells and/or cell contents of the solid body sample have been suspended or dispersed in a fluid, preferably a liquid. In the above-mentioned examples, the biological sample is typically taken from a subject. The embodiments are, however, not limited to body fluid samples or solid body samples but could also include other types of samples, in particular liquid samples, such as soil samples, food samples, water samples, etc.

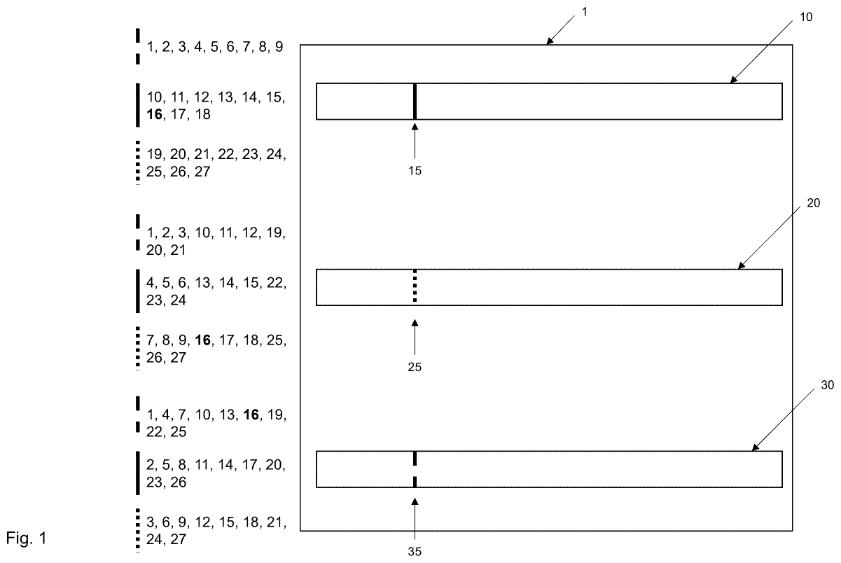

FIG. 8 is a flow chart illustrating an in vitro detection method according to the invention, see also FIGS. 1 to 3. The method comprises contacting, in step S1 and for each detection channel 10, 20, 30, 40 of N≥2 detection channels 10, 20, 30, 40, a sample with a plurality of detection molecules. According to the invention, each recognition molecule of the multiple recognition molecules binds specifically to or reacts specifically with a respective subset of at least one analyte. The plurality of recognition molecules are divided into multiple detection sets comprising multiple recognition molecules of the plurality of recognition molecules. The multiple recognition molecules of each detection set of the multiple detection sets produce a same readout signal when binding specifically to or reacting specifically with an analyte. Furthermore, the N detection channels 10, 20, 30, 40 each have different detection sets or different combinations of detection sets. The method also comprises detecting, in step S3 and for each detection channel 10, 20, 30, 40 of the N detection channels 10, 20, 30, 40, any readout signal 15, 25, 35, 45 produced by a recognition molecule binding specifically to or reacting specifically with an analyte. The method further comprises identifying, in step S4, an analyte in the sample based on a barcode representing the readout signals 15, 25, 35, 45 produced for the N detection channels 10, 20, 30, 40.

Steps S1 and S3 of FIG. 8 are performed for each detection channel 10, 20, 30, 40 of the N detection channels, which is schematically represented by the line L1 in the figure.

A “detection channel” as used herein refers to an enclosed or defined volume, in which the sample can be contacted with a plurality of detection molecules. This enclosed or defined volume is furthermore spatially separated from the other detection channel(s) 10, 20, 30, 40 of the N detection channels 10, 20, 30, 40. An illustrative example of detection channels are channels, such as microfluidic channels, in a substrate or chip. Other examples of detection channels include lateral flow channels, such as in the form of LFTs or LFIAs. Also, other types of channels, such as reaction channels in a substrate or chip, could be used as detection channels according to the embodiment. In the above-mentioned examples, the detection channels are typically in the form of elongated enclosed or defined volumes. The embodiments are, however, not limited thereto. In fact, the detection channels could have any generally shape, size or form. As further examples, the detection channels could be wells of a multi-well plate or chambers in a chamber plate, chip or substrate. The detection channels do not necessarily have to be present on the same physical device but could be separate devices enclosing a respective volume configured to house the sample and the recognition molecules. Examples of such separate devices include test tubes, such as Eppendorf® tubes, various vials, microtubes, etc. Generally, the detection channels 10, 20, 30, 40 could be any enclosed or defined volume configured to house, i.e., contain or comprise, the recognition molecules and the sample, and in which the recognition molecules can bind specifically to or react specifically with analytes.

Step S1 could be performed according to various embodiments. For instance, a portion of the sample could be added to each detection channel 10, 20, 30, 40 and where each detection channel 10, 20, 30, 40 is preloaded with recognition molecules. In another embodiment, the sample and the recognition molecules are added to the detection channels 10, 20, 30, 40, such as simultaneously or sequentially in any order. In the former case, the sample and different detection sets of recognition molecules could be mixed prior to adding the different mixtures to the detection channels 10, 20, 30, 40. Accordingly, the contact between the sample and the plurality of recognition molecules in step S1 could be performed in situ in the different detection channels 10, 20, 30, 40 or outside of the detection channels 10, 20, 30, 40. According to the invention, each recognition molecule binds specifically to or reacts specifically with a respective subset of at least one analyte. The means that each recognition molecule has the ability or capacity to bind specifically to a respective subset of at least one analyte or has the ability or capacity to react specifically with a respective subset of at least one analyte.

In an embodiment, each recognition molecule binds specifically to a respective subset of at least one analyte.

By “bind specifically to” and similar expressions it is meant that the recognition molecule in question specifically binds to the target analyte or analytes without any significant binding to other molecules. The specificity of a recognition molecule can be determined based on affinity and/or avidity. For instance, the affinity is represented, for antibodies, or antigen-binding fragments thereof, as recognition molecules, by the equilibrium dissociation constant of an antigen with the antibody (K_{D}). This K_{D} is a measure for the binding strength between an antigenic determinant, i.e., epitope, and an antigen-binding site on the antibody, or the antigen-binding fragment thereof. The lower the value of K_{D}, the stronger the binding strength between the antigenic determinant and the antibody, or the antigen-binding fragment thereof. Alternatively, the affinity can also be expressed as the equilibrium association constant (K_{A}), which is 1/K_{D}. As will be clear to the skilled person, affinity can be determined in a manner known per se, depending on the specific antigen of interest. For instance, specific binding of an antibody, or the antigen-binding fragment thereof, to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, surface plasmon resonance (SPR), biolayer interferometry (BLI) and different variants thereof known per se in the art.

Typically, antibodies, or the antigen-binding fragment thereof, will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e., with an affinity constant (K_{A}) of 10⁵ to 10¹² M⁻¹ or more, and preferably 10⁷ to 10¹² M⁻¹ or more and more preferably 10⁸ to 10¹² M⁻¹. Generally, any K_{D} value greater than 10⁻⁴ M (or any K_{A} value lower than 10⁴ M⁻¹) is considered to indicate non-specific binding.

Illustrative, but non-limiting, examples of recognition molecules that bind specifically to a subset of at least one analyte include antibodies, antigen-binding fragments thereof, aptamers, and nucleic acid probes.

Antibodies as used herein include polyclonal antibodies and monoclonal antibodies. An antigen-binding fragment of an antibody as used herein can be selected from a group consisting of a single chain or domain antibody (sdAb), such as Nanobody® molecule or variable domain of heavy chain-only (VHH) antibody, a Fv fragment, a single-chain variable fragment (scFv), a Fab fragment, a F(ab′) 2 fragment, a Fab′ fragment, a Fd fragment, a single-domain antibody (sdAb), a scFv-Fc fragment, a di-scFv fragment and a complementarity-determining region (CDR).

Aptamers are short sequences usually of nucleic acid molecules, such as DNA or RNA, which bind a specific analyte or a subset of at least one analyte. Like antibodies, they can show strong binding to their target, with little or no off-target binding. The aptamers are most commonly in the form of oligonucleotides binding specifically to proteins as targets, but can also be generated against cells, prions, bacteria and viruses. Although less common, aptamers could be in the form of peptide aptamers, which are artificial polypeptides selected or engineered to bind specific target molecules. Such peptide aptamers usually comprise one or more peptide loops of variable sequence displayed by a protein scaffold. It is also possible to have adapted that are combinations of peptides and oligonucleotides, known as tadpoles, in which peptide aptamer “heads” are covalently linked to unique sequence double-stranded DNA “tails”.

Nucleic acid probes, also referred to as hybridization probes (HPs), are nucleic acid molecules of usually 15-10.000 nucleotides long, which can be used to detect the presence of nucleic sequences in nucleic acid molecules that are complementary to the sequence of the nucleic acid probe. Illustrative, but non-limiting, examples of such nucleic acid probes include Scorpion® probes, molecular beacon probes, TaqMan® probes, LNA® probes, padlock probes, quenched probes and cycling probes. The nucleic acid probe could comprise naturally occurring nucleotides, such as DNA nucleotides and/or RNA nucleotides. The nucleic acid probe may alternatively, or in addition, comprise one or more artificial or modified nucleotides or nucleic acid analogues, such as locked nucleic acid (LNA), peptide nucleic acid (PNA), glycol nucleic acid (GNA), threose nucleic acid (TNA), hexitol nucleic acid (HNA), and/or morpholino.

In another embodiment, each recognition molecule reacts specifically with a respective subset of at least one analyte.

In such an embodiment, a recognition molecule reacts specifically with at least one analyte to produce a reaction product, the presence of which produces a readout signal in a detection channel or outside of the detection channel, which is further described herein. For instance, padlock probes (PLPs) could be used as such reaction molecules. A PLP is a long oligonucleotide, whose 5′ and 3′ ends are complementary to adjacent target sequences of a nucleic acid molecule as analyte. Upon hybridization to the target analyte, the two ends are brought into contact, allowing PLP circularization by ligation using a ligase to form a circular nucleic acid molecule. Rolling circle amplification (RCA) could then be used together with a primer to amplify the circular nucleic acid molecule into a long single stranded nucleic acid molecule comprising multiple copies of the nucleic acid sequence of the circular nucleic acid molecule. The presence of such formed long single stranded nucleic acid molecule as reaction product in a detection channel can be detected as a readout signal, for example, by hybridizing fluorescent probes or fluorophores to the reaction product. Such fluorescent probes or fluorophores can be made in, for instance, different colors such that different detection sets are assigned different colors.

Other examples of embodiments of recognition molecules that react specifically with analytes include polymerase chain reaction (PCR) primers that can be used to amplify nucleic acid molecules in PCR reactions. Other types of specific amplification methods using primers as recognition molecules include amplification with isothermal methods, such as loop-mediated isothermal amplification (LAMP), reverse transcription LAMP (RT-LAMP), and single-stranded DNA binding protein (SSB)-helicase assisted rapid PCR (SHARP). In these amplification methods, the recognition molecules are, thus, target-complementary nucleic acid probes that are used for sequence specific amplification.

The N detection channels 10, 20, 30, 40 each have different detection sets or different combinations of detection sets. FIG. 1 illustrates an example of where each detection channel 10, 20, 30 has different combinations of detection sets. Thus, each detection channel 10, 20, 30 is, in this example, associated with three detection sets. In such an embodiment, the different detection sets associated with the same detection channel 10, 20, 30 comprise different combinations of recognition molecules. Furthermore, the detection sets associated with one of the detection channels 10, 20, 30 comprise different combinations of recognition molecules as compared to the detection sets associated with the other detection channel(s) 10, 20, 30.

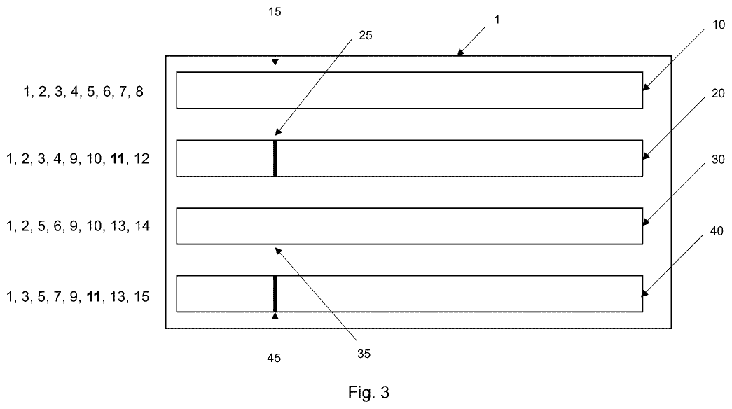

Correspondingly, if each detection channel 10, 20, 30, 40 is associated with a single respective detection set as shown in FIG. 3, then the different detection sets comprise different combinations of recognition molecules.

The multiple recognition molecules to which the sample is contacted in step S1 are divided into multiple so-called detection sets, which each comprises multiple recognition molecules. The multiple recognition molecules of a detection set produce a same readout signal when binding specifically to or reacting specifically with an analyte. For instance, 27 recognition molecules could be divided into three detection sets, each containing nine recognition molecules binding specifically to or reacting specifically with different subsets of at least one analyte. This is schematically shown in FIG. 1. For the first detection channel, the first detection set comprises recognition molecules 1-9 binding specifically to or reacting specifically with analyte nos. 1-9, respectively. The second detection set comprises recognition molecules 10-18 binding specifically to or reacting specifically with analyte nos. 10-18, respectively, and the third detection set comprises recognition molecules 19-27 binding specifically to or reacting specifically with analyte nos. 19-27. Then all recognition molecules 1-9 of the first detection set would produce the same readout signal if any of the analyte nos. 1-9 would be present in the sample for the first detection channel. For instance, a sample containing analyte no. 3 would produce the same readout signal for this first detection channel as a sample containing analyte no. 7 since the recognition molecule 3 and the recognition molecule 7 are encompassed in the same detection set and thereby produce the same readout signal for the first detection channel 10. However, a recognition molecule from the second detection set, such as recognition molecule 13, would produce a different readout signal, when binding specifically to or reacting specifically with analyte no. 13, as compared to a recognition molecule from the first detection set, such as recognition molecule 3, when binding specifically to or reacting specifically with analyte no. 3. This is schematically illustrated in FIG. 1 by the recognition molecules 1-9 of the first detection set producing a readout signal represented by a hatched line, the recognition molecules 10-18 of the second detection set producing a readout signal represented by a full line and the recognition molecules of the third detection set producing a readout signal represented by a dotted line.

According to the invention, the N detection channels 10, 20, 30, 40 each have different detection sets or different combinations of detection sets. In other words, the one or more detection sets assigned for one detection channel have different combinations of recognition molecules as compared to the detection set(s) assigned for another detection channel. With reference anew to FIG. 1, the first to third detection channels 10, 20, 30 are each assigned three detection sets comprising nine recognition molecules. The first detection set for the first detection channel 10 comprises recognition molecules 1-9 binding specifically to or reacting specifically with analyte nos. 1-9, whereas the first detection set for the second detection channel 20 comprises recognition molecules 1-3, 10-12, 19-21 binding specifically to or reacting specifically with analytes nos. 1-3, 10-12, 19-21 and the first detection set for the third detection channel 30 comprises recognition molecules 1, 4, 7, 10, 13, 16, 19, 22, 25 binding specifically to or reacting specifically with analytes nos. 1, 4, 7, 10, 13, 16, 19, 22, 25. In other words, the three detection channels comprise different combinations of the 27 recognition molecules for the three detection channels 10, 20, 30.

Step S3 of FIG. 8 comprises detecting a respective readout signal 15, 25, 35, 45 for each detection channel 10, 20, 30, 40. The readout signals 15, 25, 35, 45 are produced by a respective recognition molecule binding specifically to or reacting specifically with an analyte. An analyte in the sample can then be identified in step S4 based on a barcode representing the readout signals 15, 25, 35, 45 produced by the N detection signals 10, 20, 30, 40.

In FIG. 1, the readout signal 15 for the first detection channel 10 is a full line indicating that one of the recognition molecules from the second detection set (recognition molecules 10-18) has bound specifically to or reacted specifically with an analyte in the sample. The readout signal 25 for the second detection channel 20 is a dotted line indicating that one of the recognition molecules from the third detection set (recognition molecules 6-9, 16-18, 25-27) has bound specifically to or reacted specifically with an analyte in the sample. Finally, the readout signal 35 for the third detection channel 30 is a hatched line indicating that one of the recognition molecules from the first detection set (recognition molecules 1, 4, 7, 10, 13, 16, 19, 22, 25) has bound specifically to or reacted specifically with an analyte in the sample. The barcode will in this case, thus, be a “full line” readout signal 15, a “dotted line” readout signal 25 and a “hatched line” readout signal 35. The only recognition molecule that is present in all of the detection sets generating such a barcode is recognition molecule 16 marked in bold in FIG. 1. This means that the sample comprises analyte no. 16.

In an embodiment, the in vitro detection method of FIG. 8 comprises an additional step S2 as shown in the figure. This step S2 comprises separating, for each detection channel 10, 20, 30, 40 of the N detection channels 10, 20, 30, 40, any recognition molecule bound specifically to or reacted specifically with an analyte from recognition molecules not bound specifically with an any analyte.

This optional separation in step S2 thereby removes non-bound or non-reacted recognition molecules for a detection channel 10, 20, 30, 40 from the recognition molecules bound to or reacted specifically with an analyte. The separation in step S2 could be performed according to various embodiments and based on different physical characteristics of the complex or the formed reaction product. For instance, the analyte with bound recognition molecule or the reaction product produced by a recognition molecule reacting specifically with an analyte could have a different, typically larger, size as compared to non-bound or non-reacted reaction molecules. In such a case, a size-based separation could be used in step S2. For instance, a size-based filtration could be used in step S2. Various such size-based filtrations are available for separating recognition molecules, including filters, gel filtration, etc. Other examples of size-based separation in step S2 is centrifugation and binding to a solid phase having preference for nucleic acid molecules of a given length and/or sequence. The separation in step S2 does not necessarily have to be based on size but could also be based on affinity to the complex between a recognition molecule and an analyte or a reaction product to thereby immobilize the recognition molecule with bound analyte or the reaction product to a solid substrate (Lee et al., A Lateral Flow Assay for Nucleic Acid Detection Based on Rolling Circle Amplification Using Capture Ligand-Modified Oligonucleotides, BioChip Journal 2022; Jain et al., Padlock probe-based rolling circle amplification lateral flow assay for point-of-need nucleic acid detection, Analyst 2021, 146:4340-4347; Jauset-Rubio, Ultrasensitive, rapid and inexpensive detection of DNA using paper based lateral flow assay, Scientific Reports 2016, 6:37732).

Hence, in an embodiment, step S2 comprises separating, for each detection channel 10, 20, 30, 40 of the N detection channels 10, 20, 30, 40, any recognition molecule bound specifically to or reacted specifically with an analyte from recognition molecules not bound specifically to or reacted specifically with any analyte by at least one of size-based filtration, centrifugation and affinity binding to a solid substrate.

In an embodiment, step S1 comprises adding the sample to N lateral flow channels 10, 20, 30, 40 preloaded with the plurality of recognition molecules. In such an embodiment, step S2 comprises separating, for each lateral flow channel 10, 20, 30, 40 of the N lateral flow channels 10, 20, 30, 40, any recognition molecule bound specifically to or reacted specifically with an analyte from recognition molecules not bound specifically to or reacted specifically with any analyte.

Such lateral flow channels 10, 20, 30, 40 typically comprises a sample pad, to which the sample is added in step S1. The lateral flow channels 10, 20, 30, 40 also comprise a respective conjugate pad comprising the preloaded recognition molecules and a nitrocellulose membrane, which presents capillary action to achieve a capillary flow of the sample from the sample pad through the conjugate pad and along the nitrocellulose membrane towards a downstream wicking pad. Any analyte in the sample may then be bound to or react with recognition molecules as the sample and thereby any analytes present therein are flowing through the conjugate pad. The recognition molecules bound specifically to or reacted specifically with the analyte can then be captured in the nitrocellulose membrane and thereby separated from non-bound or non-reacted recognition molecules that are flowing further along the nitrocellulose membrane towards the wicking pad.

In an embodiment, step S1 comprises adding the sample to the N lateral flow channels 10, 20, 30, 40 preloaded with a plurality of recognition molecules comprising a respective detectable moiety and a plurality of recognition molecules comprising an epitope tag. In such an embodiment, each lateral flow channel 10, 20, 30, 40 comprises affinity molecules attached to a solid support and binding specifically to the epitope tag. A pair of a recognition molecule comprising a respective detectable moiety and a recognition molecule comprising the epitope tag binds specifically to a respective subset of at least one analyte.

Hence, in this embodiment, a pair of recognition molecules binds specifically to a respective subset of at least one analyte. In such a case, one of the recognition molecules in the pair comprises a detectable moiety and the other recognition molecule in the pair comprises the epitope tag. The recognition molecules comprising epitope tags can then be captured by the affinity molecules attached to the solid support, such as nitrocellulose membrane. In such a case, complexes between a recognition molecule with a detectable moiety, an analyte and a recognition molecule with the epitope tag will be captured by the affinity molecules and a readout signal can be produced based on the detectable moiety. Non-bound recognition molecules with detectable moieties will flow the detection linear or area with immobilized affinity molecules and further towards the wicking pad.

In this embodiment, different detectable moieties are used by the recognition molecules in the different detection sets.

The detection moiety could be any moiety that could be detected to produce a readout signal. Illustrative, but non-limiting, examples of such detection moieties include nanoparticles, such as gold nanoparticles (GNPs, AuNPs), quantum dots (QDs), colored latex beads, lyposme-encapsulated dyes, fluorescence labels or tags, chemiluminescence labels or tags, radiolabels or radiotags, enzymatic labels, etc.

The affinity molecules attached or immobilized to the solid support could, for instance, be antibodies, or antigen-binding fragments thereof. In such a case the epitope tag comprised in some of the recognition molecules is preferably a peptide epitope, to which the antibodies, or the antigen-binding fragments thereof, bind specifically. In another embodiment, the affinity molecules could be nucleic acid molecules with the epitope tag in the form of a nucleic acid molecule that is can be hybridized to the nucleic acid molecules attached to the solid support by base-pairing.

In this embodiment, each detection set comprises multiple pairs of recognition molecules, wherein the recognition molecules of such a pair both bind specifically to or react specifically with a subset of at least one analyte. For instance and with reference to FIG. 1, the first detection set then comprises nine pairs of recognition molecules. The first pair comprises recognition molecules 1a, 1b both binding specifically to or reacting specifically with analyte no. 1. The first recognition molecule 1a of the pair comprises the detectable moiety and the second recognition molecule 1b of the pair comprises the epitope tag. The same epitope tags could be used for all recognition molecules in the detection set, or indeed for all recognition molecules of all detection sets. Furthermore, the same detection moiety is used for the moiety-containing recognition molecules 1a-9a of the first detection set, whereas other detection moieties are used for the moiety-containing recognition molecules 10a-18a, 19a-27a of the second and third detection sets.

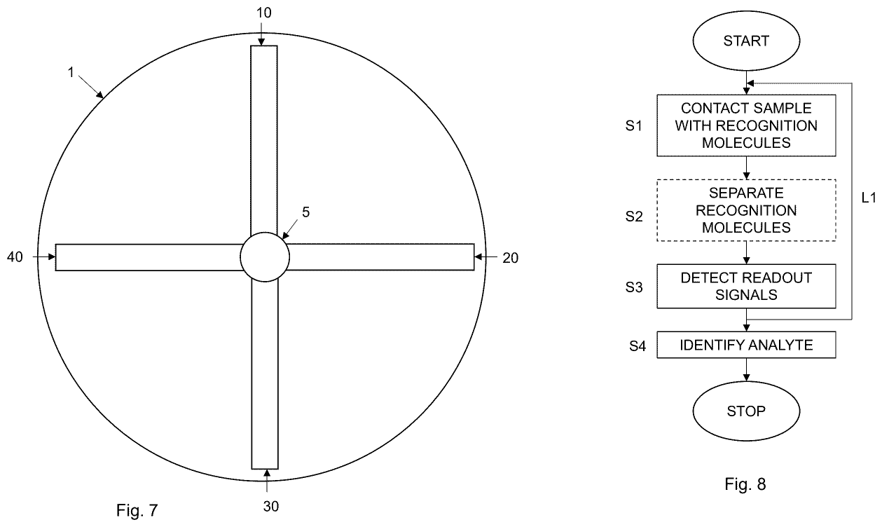

FIG. 7 schematically illustrates a substrate in the form of a disc 1 comprising multiple lateral flow devices or other forms of detection channels 10, 20, 30, 40. In such a case, the disc 1 can comprise a common sample inlet 5, to which the sample is added and then further divided into the different lateral flow devices or other forms of detection channels 10, 20, 30, 40.

The above-described separation step S2 is optional and in various embodiments the readout signals 15, 25, 35, 45 can be detected in step S3 without the need for any separation. For instance, reaction products in the form of rolling circle amplification products (RCPs) typically form aggregates or complexes of long nucleic acid molecules. In such a case, multiple recognition molecules, such as in the form of fluorescent probes or fluorophores, could bind to the RCPs and the readout signal is readily visible by the many recognition molecules binding to such RCP aggregates or complexes without the need to any separation of non-bond recognition molecules.

In an embodiment, each recognition molecule of the plurality of recognition molecules binds specifically to or reacts specifically with a respective subset of at least one analyte that is different than the respective subset of at least one analyte, to which other recognition molecules of the plurality of recognition molecules binds specifically or with which other recognition molecules of the plurality of recognition molecules reacts specifically.

In an embodiment, the N detection channels 10, 20, 30, 40 each have different combinations of recognition molecules in different detection sets as shown in FIG. 1. In such an embodiment, for each detection channel 10, 20, 30, 40 of the N detection channels 10, 20, 30, 40, the multiple recognition molecules of each detection set of the multiple detection sets produce a readout signal that is different than readout signal(s) produced by recognition molecules of the other detection set(s) of the multiple detection sets. For instance, the recognition molecules of the first detection set for the first detection channel 10 produce a readout signal 15 represented by hatched line in FIG. 1. The recognition molecules of the second detection set for the first detection channel 10 produce a readout signal 15 represented by full line, whereas recognition molecules of the third detection set for the first detection channel 10 produce a readout signal 15 represented by dotted line.

As discussed in the foregoing, each recognition molecule binds specifically to or reacts specifically with a respective subset of at least one analyte. In an embodiment, the subset comprises multiple analytes. Hence, in such an embodiment, each recognition molecule, or at least a portion of the plurality of recognition molecules, binds specifically to or reacts specifically with at least two analytes. For instance, the at least two analytes could be analytes of a same class or group of analytes sharing a common epitope or nucleic acid sequence to which a recognition molecule could bind specifically or with which a recognition molecule could react specifically. This could, for example, be nucleic acid probes recognizing conserved regions in 16S ribosomal RNA for a specific family of bacteria or antibodies recognizing different types of beta-lactamase conferring resistance to beta-lactam antibiotics. Hence, in such an embodiment, at least some of the recognition molecules are broad-specific recognition molecules capable of detecting several analytes in a group or a class. All of the plurality of recognition molecules could be such broad-specific recognition molecules. Alternatively, merely a portion or subset of one or more of the plurality of recognition molecules are broad-specific recognition molecules with the remaining recognition molecules binding specifically to or reacting specifically with a respective analyte rather than a group or class of multiple analytes.

In another embodiment, each recognition molecule of the plurality of recognition molecules binds specifically to or reacts specifically with a respective analyte. Hence, in this embodiment, each recognition molecule merely binds specifically to or reacts specifically to a respective analyte rather than to a group or class of multiple analytes. There is, thus, a one-to-one relationship between the number of recognition molecules and the number of analytes that can be detected.

In an embodiment, the number of different recognition molecules, with which the sample is contacted in step S1, is larger than N. This means that the in vitro detection method can detect more potential analytes than the number of detection channels 10, 20, 30, 40. For instance, as shown in FIG. 1, 3³=27 recognition molecules could be used to detect 27 potential analytes by merely using three detection channels 10, 20, 30. Furthermore, if the readout signal is the presence or absence of a signal then it is possible to detect up to 2^{N}−1 different analytes using up to 2^{N}−1 recognition molecules. For instance, 1023 primer pairs could be used as recognition molecules in each of 10 recognition channels to thereby detect one out of 1023 genomic mutations in sample comprising such genomic DNA.

In an embodiment, each analyte has a unique barcode. Hence, in such an embodiment, each analyte of the potential analytes in the sample produce a unique combination of readout signals for the N detection channels 10, 20, 30, 40 and thereby a unique barcode. In other words, two different analytes have different barcodes when running the in vitro detection method.

Although less preferred, a group or class of similar analytes could be assigned a same barcode to indicate that the sample contain an analyte of a specific class or type although the exact identity of the analyte is not determined. For instance, presence of different resistance genes against a given antibiotic could produce the same combination of readout signals and the same barcode to indicate the presence of resistance against the given antibiotic. Correspondingly, bacteria of the same family, such as Enterobacteriaceae, could, when present in a sample, generate the same barcode. Another example is that different mutations in the same gene may generate the same barcode.

In an embodiment, step S1 in FIG. 8 comprises contacting, for each detection channel number n 10, 20, 30, 40 of the N detection channels 10, 20, 30, 40, the sample with Mₙ recognition molecules. In this embodiment, n=1 . . . N and each Mₙ is an integer equal to or larger than 2. In a preferred embodiment, each Mₙ is larger than N. In this embodiment, the sample is contacted with M₁ recognition molecules in the first detection channel 10, M₂ recognition molecules in the second detection channel 20, and so forth. These numbers Mₙ could be the same, i.e., Mₙ=T, wherein T is a defined number. The embodiments are, however, not limited thereto. In an embodiment, at least two of the numbers Mₙ may be different, i.e., Mᵢ≠Mⱼ for some i≠j, wherein i, j∈[1, N]. In a preferred embodiment, the numbers Mₙ are the same, i.e., the sample is contacted with the same number of recognition molecules in the N detection channels 10, 20, 30, 40.

In an embodiment, the plurality of detection molecules in each detection channel number n are divided into Cₙ≥2 detection sets, wherein n=1 . . . N. In such an embodiment, step S4 comprises identifying the analyte in the sample out of up to

$${C}_{1} \times … \times {C}_{N} = {\prod }_{n = 1}^{N} {C}_{n}$$

possible or potential analytes based on the barcode representing the readout signal 15, 25, 35, 45 produced for the N detection channels 10, 20, 30, 40. For instance, if the detection molecules are divided into C detection sets in each detection channel, i.e., each Cₙ=C, then up to C^{N} potential analytes could be identified by the in vitro detection method, where each analyte is identified by one detection set in each detection channel. If we do not require that each analyte is identified by one detection set in each detection channel, i.e., that the absence of signal is also used for coding then the number of analytes that can be identified increase to (C+1)^{N}−1.

In a particular embodiment, step S4 comprises identifying the analyte in the sample out of

$${\prod }_{n = 1}^{N} ( {C}_{n} + 1 ) - 1 or {\prod }_{n = 1}^{N} {C}_{n}$$

potential analytes, such as out of (C+1)^{N}−1 or C^{N} potential analytes based on the barcode representing the readout signal 15, 25, 35, 45 produced for the N detection channels 10, 20, 30, 40. Such an example is shown in FIG. 1 with C₁=C₂=C₃=C=3 and N=3 to thereby be able to identify up to 3³=27 potential analytes. As indicated above, all Cₙ may be the same, i.e., C, for n=1 . . . N. However, the embodiments are not limited thereto. Hence different numbers of detection sets could be used for different detection channels, i.e., Cᵢ≠Cⱼ for some i≠j, wherein i, j∈[1, N].

In an embodiment, the plurality of detection molecules in each detection channel number n 10, 20, 30, 40 are divided into Cₙ=2 detection sets, for instance C≥2 detection sets. In such an embodiment, step S1 comprises contacting, for each detection channel 10, 20, 30, 40 of the N detection channels 10, 20, 30, 40, the sample with a plurality of recognition molecules out of up to

$${\prod }_{n = 1}^{N} ( {C}_{n} + 1 ) - 1 or {\prod }_{n = 1}^{N} {C}_{n}$$

recognition molecules, for instance C^{N} recognition molecules. For instance, in total

$${\prod }_{n = 1}^{N} ( {C}_{n} + 1 ) - 1 or {\prod }_{n = 1}^{N} {C}_{n}$$

different recognition molecule species could be used in the in vitro detection method, such as (C+1)^{N}−1 or C^{N} or different recognition molecule species could be used in the in vitro detection method.

In such an embodiment, step S1 preferably comprises contacting, for each detection channel number n 10, 20, 30, 40 of the N detection channels 10, 20, 30, 40, the sample with up to

$${\prod }_{n = 1}^{N} ( {C}_{n} + 1 ) - 1 or {\prod }_{n = 1}^{N} {C}_{n}$$

recognition molecules, such as (C+1)^{N}−1 or C^{N} recognition molecules, wherein n=1 . . . N. In such a preferred embodiment, step S3 comprises detecting, for each detection channel number n 10, 20, 30, 40 of the N detection channels 10, 20, 30, 40, a readout signal cₙ 15, 25, 35, 45 produced by a recognition molecule binding specifically to or reacting specifically with an analyte. Each Cₙ represents a readout out of Cₙ possible readouts for detection channel number n, such as out of C possible readouts. Step S4 then comprises, in this preferred embodiment, identifying the analyte in the sample based on the barcode [c₁, . . . , c_{N}].

The following provides illustrative examples of using

$${\prod }_{n = 1}^{N} ( {C}_{n} + 1 ) - 1$$

or (C+1)^{N}−1 recognition molecules.

Example 1 (binary readout signals) involves having three detection channels, i.e., N=3, with the following detection sets: one detection set with recognition molecules 1, 2, 3, 4 for the first detection channel, one detection set with recognition molecules 1, 2, 5, 6 for the second detection channel and one detection set with recognition molecules 1, 3, 5, 7 for the third detection channel. In this illustrative example, N=3 and C₁=C₂=C₃=1 and the number of recognition molecules is, thus, (1+1)³−1=7.

Example 2 involves having two detection channels, i.e., N=2, with the following detection sets: a first detection set with recognition molecules 1, 3, 6 and a second detection set with recognition molecules 2, 4, 5 for the first detection channel and a first detection set with recognition molecules 2, 3, 7 and a second detection set with recognition molecules 1, 4, 8 for the second detection channel. In this illustrative example, N=2 and C₁=C₂=2 and the number of recognition molecules is, thus, (2+1)²−1=8.

With reference to FIG. 1, the three readout signals c₁, c₂, c₃ each represents one out of C=3 possible readouts (hatched, full or dotted lines). The barcode is then the combination of these readout signals c₁, c₂, c₃, i.e., full, dotted and hatched lines in the example shown in FIG. 1.

In an embodiment, the N detection channels 10, 20, 30, 40 each have different combinations of detection sets, see FIG. 1. For each detection channel 10, 20, 30, 40 of the N detection channels 10, 20, 30, 40, the multiple recognition molecules of each detection set of the multiple detection sets produce a color signal that is different than color signal(s) produced by recognition molecules of other detection set(s) of the multiple detection sets. For instance, the different lines as shown in FIG. 1 could represent different colors, such as red, green and blue. In such an embodiment, step S3 comprises detecting, for each detection channel 10, 20, 30, 40 of the N detection channels 10, 20, 30, 40, a color signal 15, 25, 35, 45 produced by the recognition molecule binding specifically to or reacting specifically with the analyte. Step S4 then comprises identifying the analyte in the sample based on a barcode representing the color signals 15, 25, 35, 45 produced for the N detection channels 10, 20, 30, 40.

The embodiments are, however, not limited to readouts in the form of different colors. This concept could be applied to fluorescence at different wavelengths or wavelength intervals, using different types of nanoparticles, labeled nucleic acid probes, etc.

In other embodiment, the readout signal is a binary readout signal, i.e., being one out of two different types of signals, such as presence of a signal versus absence of a signal. FIG. 3 illustrates such an approach. In such an embodiment, step S3 comprises detecting, for each detection channel 10, 20, 30, 40 of the N detection channels 10, 20, 30, 40, a binary readout signal 15, 25, 35, 45 produced by the recognition molecule binding specifically to or reacting specifically with the analyte. In such an embodiment, step S4 comprises identifying the analyte in the sample based on a barcode representing the binary readout signals 15, 25, 35, 45 produced for the N detection channels 10, 20, 30, 40.

In FIG. 3, each detection channel 10, 20, 30, 40 is associated with a respective detection set of recognition molecules. Different such combinations of recognition molecules are used for the different detection channels 10, 20, 30, 40. The readout signals are, in this embodiment, presence (full line) or absence (no line) of a readout in a detection channel. In FIG. 3, the barcode will be absence, presence, absence and presence of a readout. The analyte present in the sample will thereby be analyte no. 11 as marked in bold in the figure. In this embodiment, at most 2^{N}−1 different analytes can be detected.

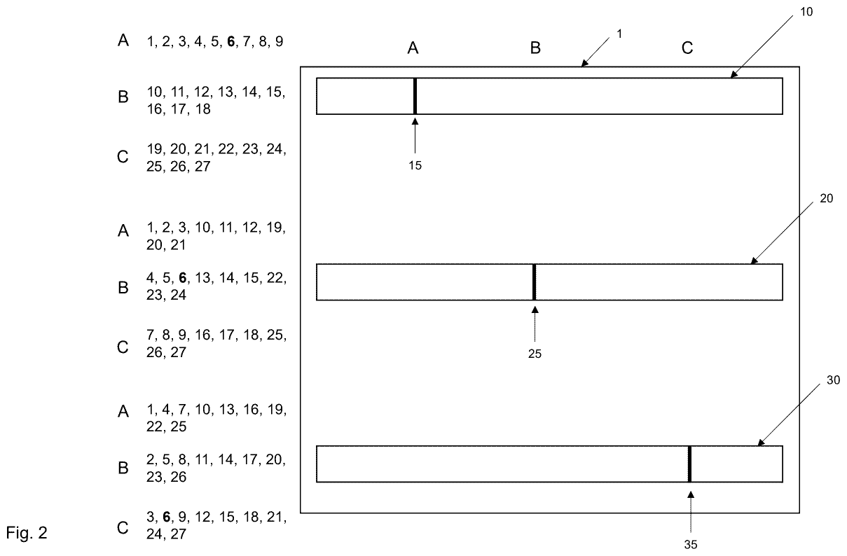

FIG. 2 illustrates a further embodiment of different readouts for the in vitro detection method. In this embodiment, step S3 comprises detecting, for each detection channel 10, 20, 30 of the N detection channels 10, 20, 30, a readout signal 15, 25, 35 representing a spatial location of a readout in the detection channel 10, 20, 30 and produced by the recognition molecule binding specifically to or reacting specifically with the analyte. Step S4 comprises, in this embodiment, identifying the analyte in the sample based on a barcode representing the spatial locations of the readouts produced for the N detection channels 10, 20, 30.

In FIG. 2 and the first detection channel 10, the recognition molecules are divided into three detection set. The first detection set comprises recognition molecules 1-9 and produces a readout at spatial position A in the first detection channel 10 if the sample comprises any of the analyte nos. 1-9. Correspondingly, the second detection set comprises recognition molecules 10-18 and produces a readout at spatial position B in the first detection channel 10 if the sample comprises any of the analyte nos. 10-18 and the third detection set comprises recognition molecules 19-27 and produces a readout at spatial position C in the first detection channel 10 if the sample comprises any of the analyte nos. 19-27. In the particular example of FIG. 2, the barcode is A, B and C indicating that the sample comprises analyte no. 6 marked in bold in the figure.

Step S3 of FIG. 8 comprises detecting a readout signal 15, 25, 35, 45 for each detection channel 10, 20, 30, 40 of the N detection channels 10, 20, 30, 40. The detection of the readout signal does not necessarily have to take place in the N detection channels 10, 20, 30, 40. For instance, the complex between a recognition molecule binding specifically to an analyte or the reaction product as produced between a recognition molecule reacting specifically to an analyte in a detection channel 10, 20, 30, 40 could be detected outside of the detection channel 10, 20, 30, 40, such as in a position downstream of the detection channel 10, 20, 30, 40 or indeed elsewhere separate from the detection channel 10, 20, 30, 40. As an illustrative example, a reaction product as produced in a PCR reaction could be captured in an agarose gel, where detection of the reaction product is taking place. Other examples include detecting reaction products by fluorescence microscopy or cell sorting.

In such a case, the complexes and reaction products from the N detection channels 10, 20, 30, 40 could be kept physically separated to thereby obtain up to N physically separated detection signals. It is, however, possible to mix or pool the complexes and reaction products from the N detection channels 10, 20, 30, 40, for instance, if each reaction molecule comprises a respective channel-specific barcode or signal. In such a case, it is still possible to differentiate the up to N detection signals even following pooling due to the presence of channel-specific barcodes or signals. As used herein, a channel-specific barcode or signal indicates a barcode or signal that is specific for a detection channel 10 of the N detection channels 10, 20, 30, 40 and is thereby different from the corresponding channel-specific barcodes or signals of the other detection channels 20, 30, 40 of the N detection channels 10, 20, 30, 40.

In an embodiment, the analytes are different proteins. In such an embodiment, the recognition molecules are selected from the group consisting of aptamers, antibodies, and antigen-binding fragments thereof. In another embodiment, the analytes are different nucleic acid molecules. In such an embodiment, the recognition molecules are nucleic acid probes.

Herein, various applications of the in vitro detection method will be further exemplified.

In an application, the presence of an RNA molecule from a pathogen in sample is to be determined. The RNA molecule can be identified by cDNA synthesis using standard protocols followed by target specific PCR, direct amplification by isothermal method, such as LAMP, or SSB-Helicase Assisted Rapid PCR, or padlock probe hybridization, ligation and rolling circle amplification. The recognition molecules are in these cases the target-complementary nucleic acid probes that are used for sequence specific amplification. The probes are mixed in different sets in different detection channels, which for example can be different test tubes or reaction chambers in a microfluid device. If, for example, seven different analytes (A, B, . . . , G) should be recognized in three tubes, the corresponding probes (pA, pB, . . . , pG), can be mixed as follows:
tube 1: pA, pB, pC, pD
tube 2: pA, pB, pE, pF
tube 3: pA, pC, pE, pG

Successful recognition of the RNA molecule by a recognition molecule results in a detectable reaction product, such as a DNA band on a gel, a color change in a tube due to staining of a DNA product, for example by SYBR® green, binding of a fluorescent oligonucleotide probe, a molecular beacon that makes a florescent signal after binding or quenching in response to binding the reaction product (Kurata et al., Fluorescent quenching-based quantitative detection of specific DNA/RNA using a BODIPY® FL-labeled probe or primer, Nucleic Acids Research 2001, 29(6): e34; Gadkar et al., Real-time Detection and Monitoring of Loop Mediated Amplification (LAMP) Reaction Using Self-quenching and De-quenching Fluorogenic Probes Scientific Reports 2018, 8:5548).

In another application, the in vitro detection method is used to detect presence of a protein in a sample. The protein can be recognized by many different affinity reagents, such as antibodies, Nanobody® molecules, designed ankyrin repeat proteins, scFvs, or aptamers. If specificity is critical one can, for example, use a proximity ligation assay (PLA) where the proximal binding of two different antibodies results in a DNA circle that can be amplified in an RCA reaction (Fredriksson et al., Protein detection using proximity-dependent DNA ligation assays, Nature Biotechnology 2002, 20:473-477). In the case of PLA, the reaction product can be detected as the nucleic acid products above.

In a further application, a pair of antibodies are used per analyte. One of these antibodies are bound to a detectable moiety, such as a gold particle, that is associated to the analyte if it is present in the sample. The other antibody has an epitope tag that it shares with the other antibodies in the same detection set. This epitope tag is then captured at a detection line, such as in a lateral flow device.

In yet another application, the in vitro detection method is used to detect the presence of a rare point mutation in genomic DNA. In such a case it is possible to use a ligase mediated gene detection that can be made very sensitive to a single point mutation (Landegren et al., A ligase-mediated gene detection technique, Science 1988, 241(4869): 1077-1080) since the ligation reaction is very sensitive to mismatches in the bases next to the ligation site. The ligated products can be amplified with thermocycling or an iso-thermal method. The ligated probes can be part of a padlock probe so that the ligated product can be amplified with RCA. For example, three different padlock probes can be used to detect three different mismatches in a single position and later be read out by the same florescent probes binding to a sequence in the padlock probes' backbone that is shared by the set of padlock probes that should give the same readout in that detection channel. The coding schemes can be such that a point mutation always gives a positive readout in six different detection channels out of 12 detection channels, and otherwise no signal. This implies that it is possible to detect Bin(12, 6)=924 different point mutations. If there are more than one position that is mutated this would show up as more than six positive detection channels. The parallel use of many padlock probes to detect mutations is fully feasible as previously described Banér et al., Parallel gene analysis with allele-specific padlock probes and tag microarrays, Nucleic Acids Research 2003, 31(17): e103.

Detection of rare mutations can, for instance, be used for prenatal screening of blood from the mother, which contain small amounts of fetal cells. It is also possible to screen patient blood for mutation signatures of cancer cells to be used as early detection or relapse monitoring. For instance, by using five detection channels and four detection sets with different colors it is possible to identify 4⁵=1024 different mutations in five reactions and using flow cytometry for readout (Chen et al. Nature communications, (2022) 13:4033). As an example, four detection sets of padlock probes per detection channel could be used, where the padlock backbone sequence for padlock probes of the same detection set shares a sequence that can that can be targeted by fluorescent oligoes. In this way the padlock probes from a detection set can get a unique color that is different from other detection sets in the same channel. In such an example, getting no signals means that there was no mutation, whereas getting mixed colors for any detection channel implies that more than one mutation is present.

The in vitro detection method can also be designed to simultaneously detect a bacterial pathogen and resistance genes, for example by sensitive bacterial detection against 16s rRNA using species specific probes (Jarvius et al., Digital quantification using amplified single-molecule detection 2006, 3: 725-727) in combination with detection of resistance proteins, such as beta-lactamase conferring resistance to beta lactam antibiotics, or antibiotic target mutations, such as gyrase mutations conferring resistance to fluoroquinolones.

As discussed in the foregoing, different types of readout signals are possible according to the embodiments. These types include different colors of recognition molecules comprising different dyes, such as colored latex beads or lyposme-encapsulated dyes, different fluorophores or fluorescence labels or tags capable of producing different fluorescent signals for the recognition molecules, different chemiluminescent signals from recognition molecules comprising different chemiluminescence labels or tags, recognition molecules attached to different nanoparticles, such as gold nanoparticles or quantum dots, or different radiolabels or radiotags. The readouts could alternatively be in the form of formation of cloudiness, phase separation, solidification or clotting in the detection channels, in particular as binary readout signals.

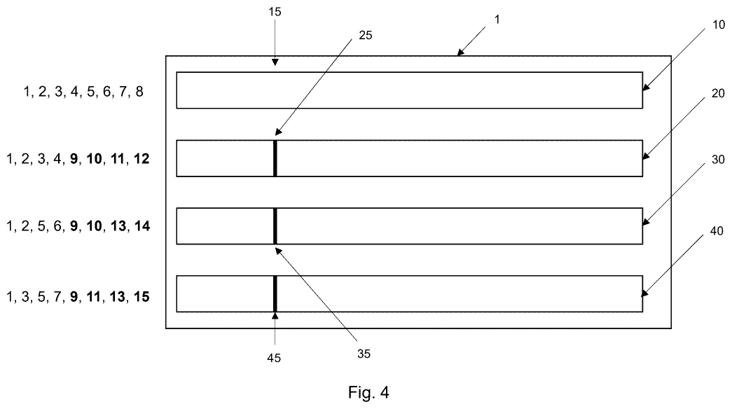

A limitation of binary readout signal as shown in FIG. 3 is that it is generally not possible to determine if there are multiple analytes present in the same sample. For example, in FIG. 4, it is not possible to know if there is a single analyte no. 9 in the sample or a mixture of two or more analytes out of analytes nos. 9 to 15, such as a mixture of analyte no. 11 and 14. A solution to such a problem is to make the barcode coding such that there are always a predefined number M positive signals in the N detection channels for a given analyte. If there are then more than M positive signals in the N detection channels, we know that the sample is a mixed sample comprising two or more different analytes. The number of unique analytes that can be identified in such an embodiment is Bin (N, M), e.g., with N=6 channels and M=3 20 analytes can be identified, whereas N=7 and M=3 enable detection of 35 analytes and N=8 and M=4 enable detection of 70 analytes. Bin (N, M) represents the binomial coefficient

$$( \frac{N}{M} ) = \frac{N !}{M ! ( N - M ) !} .$$

Hence, in an embodiment, a predefined number M<N of the readout signals 15, 25, 35, 45 representing the barcode have a respective predefined readout, such as positive signal. In such an embodiment, step S4 preferably comprises identifying a mixture of at least two different analytes in the sample based on a barcode with more than M readout signals 15, 25, 35, 45 having the respective predefined readout.

For instance, six recognition molecules 1-6 could produce the following barcodes, wherein 1 represents positive readout and 0 represents no readout:
1: (1, 1, 0, 0)
2: (1, 0, 1, 0)
3: (1, 0, 0, 1)
4: (0, 1, 1, 0)
5: (0, 1, 0, 1)
6: (0, 0, 1, 1)

Then if the readout signals 15, 25, 35, 45 for the four detection channels are 10, 20, 30, 40, for instance, 1, 1, 1, 0 then the sample comprises a mixture of analyte nos. 1 and 2, 1 and 4, 2 and 4, or indeed 1, 2 and 4.

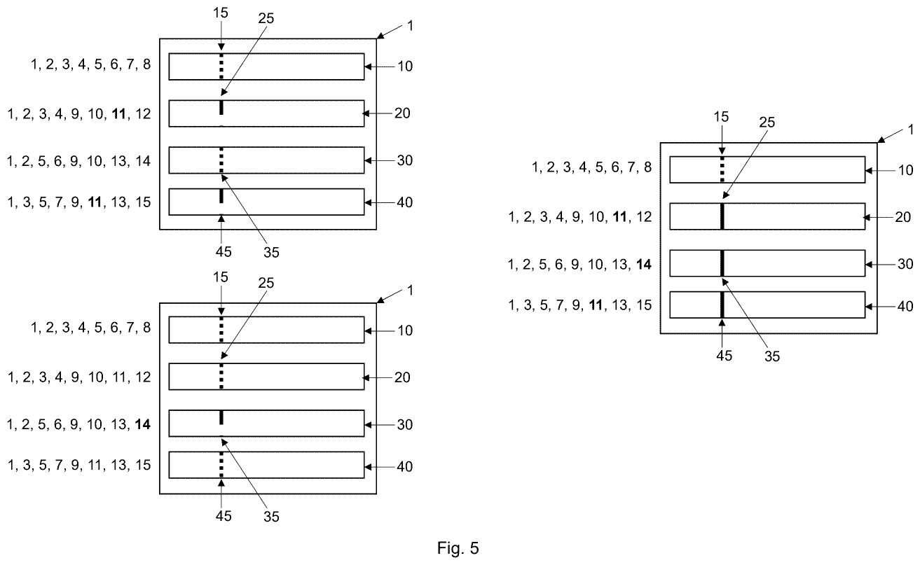

If, instead, each analyte results in a readout signal 15, 25, 35, 45 of colors in each detection channel 10, 20, 30, 40 then it is also possible to detect if there are multiple analytes present in the sample. In the example shown in FIG. 5, two different colors are represented by dotted lines (such as representing blue) and hatched lines (such as representing red). A combination of these two colors (purple) is represented by a full line. To the left of FIG. 5, the barcodes for analyte nos. 11 and 14 are shown, i.e., barcode (blue, red, blue, red) or (dotted, hatched, dotted, hatched) indicates presence of analyte no. 11 and barcode (blue, blue, red, blue) or (dotted, dotted, hatched, dotted) indicates analyte no. 14. A sample comprising a mixture of analyte nos. 11 and 14 is shown to the right and would result in a barcode (blue, purple, purple, purple) or (dotted, full, full, full).

Hence, in an embodiment, the multiple recognition molecules of each detection set of the multiple detection sets produce a predefined color signal that is different than predefined color signal(s) produced by recognition molecules of other detection set(s) of the multiple detection sets. In this embodiment, step S3 comprises detecting, for each detection channel 10, 20, 30, 40 of the N detection channels 10, 20, 30, 40, a color signal 15, 25, 35, 45 produced by the recognition molecule binding specifically to or reacting specifically with the analyte. Step S4 comprises, in this embodiment, identifying a mixture of at least two different analytes in the sample if the color signal 15, 25, 35, 45 produced for at least one detection channel 10, 20, 30, 40 is different than any of the predefined color signals. With reference to the example shown in FIG. 5, the predefined colors could be red and blue with a color different than red and blue being purple. Further, if the predefined colors are red, blue and yellow, mixtures of these colors, such as purple, green, orange, could indicate presence of a mixture of at least two analytes in the sample.

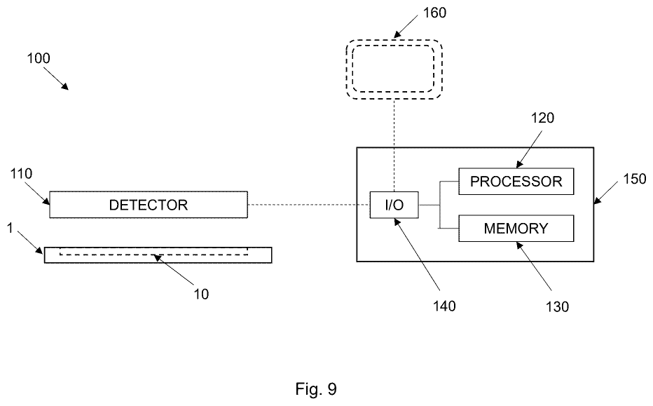

Another aspect of the invention relates to an in vitro detection device 100, see FIG. 9. The device 100 comprises N≥2 detection channels 10, 20, 30, 40 and a plurality of recognition molecules. Each recognition molecule of the plurality of recognition molecules binds specifically to or reacts specifically with a respective subset of at least one analyte. The plurality of recognition molecules are divided into multiple detection sets comprising multiple recognition molecules of the plurality of recognition molecules. The multiple recognition molecules of each detection set of the multiple detection sets produce a same readout signal when binding specifically to or reacting specifically with an analyte. The N detection channels 10, 20, 30, 40 each have different detection sets or different combinations of detection sets. Each analyte out of a plurality of possible analyte is assigned a unique barcode representing readout signals 15, 25, 35, 45 produced for the N detection channels 10, 20, 30, 40. Each readout signal 15, 25, 35, 45 of the N readout signals 15, 25, 35, 45 is produced by a recognition molecule binding specifically to or reacting specifically with an analyte for each detection channel 10, 20, 30, 40 of the N detection channels 10, 20, 30, 40.

The multiple detection channels 10, 20, 30, 40 could be in the form of separate detection channels 10, 20, 30, 40, such as separate test tubes, or could be present in a same substrate, chip or device 1, such as (microfluidic) channels or chambers in a substrate or chip, or wells in a multi-well plate.

In an embodiment, the in vitro detection device 100 also comprises a detector 110 configured to detect, for each detection channel 10, 20, 30, 40 of the N detection channels 10, 20, 30, 40, a readout signal 15, 25, 35, 45 produced by a recognition molecule binding specifically to or reacting specifically with an analyte.

The type of detector 110 is preferably selected based on the particular readout signal, such as light detector or camera for a color readout signal or nanoparticles, a fluorescence detector for a fluorescent readout signal, a detector of transmitted light for turbidity, etc.

The detector 110 could be configured to detect the readout signals 15, 25, 35, 45 in the N detection channels 10, 20, 30, 40 or outside of the N detection channels 10, 20, 30, 40 as disclosed in the foregoing.

In an embodiment, the in vitro detection device 100 further comprises a processor 120 configured to process the readout signals 15, 25, 35, 45 and determine an analyte identifier identifying an analyte out of the plurality of analytes based on a barcode representing the readout signals 15, 25, 35, 45 produced for the N detection channels 10, 20, 30, 40.

FIG. 9 schematically illustrates a processing device 150, such as computer, of the in vitro detecting device 1. Such a processing device 150 then comprises the processor 120 and a memory 130 that can be used to determine the analyte identifier. In such an embodiment, the determination of the analyte identifier could be implemented in a computer program, which is loaded into the memory 130 for execution by the processor 120 of the processing device 150. The processor 120 and the memory 130 are interconnected to each other to enable normal software execution. An input and output (I/O) unit 140 is preferably connected to the processor 120 and/or the memory 130 to enable reception of readout signals from the detector 110.

The I/O unit 140 could then be wirelessly connected to the detector 110, such as implemented in the form of a receiver, a transmitter-receiver pair or a transceiver. Alternatively, the I/O unit 140 could be connected by a wire to the detector 110 and thereby be in the form of an I/O port.

The term processor 120 should be interpreted in a general sense as any circuitry, system or device capable of executing program code or computer program instructions to perform a particular processing, determining or computing task. The processor 120 is, thus, configured to perform, when executing the computer program, well-defined processing tasks such as those described herein.

The processor 120 does not have to be dedicated to only execute the above-described steps, functions, procedure and/or blocks, but may also execute other tasks.

An optional display 160 could be wirelessly connected to the processing device 150, through the I/O unit 140, or is connected to the I/O unit 140 by a wire. The analyte identifier as determined by the processor 120 could then presented to a user on the display 160.

A further aspect of the invention relates to a kit for the in vitro detection method. The kit comprises N≥2 detection channels 10, 20, 30, 40 and a plurality of recognition molecules. Each recognition molecule of the plurality of recognition molecules binds specifically to or reacts specifically with a respective subset of at least one analyte. The plurality of recognition molecules are divided into multiple detection sets comprising multiple recognition molecules of the plurality of recognition molecules. The multiple recognition molecules of each detection set of the multiple detection sets produce a same readout signal when binding specifically to or reacting specifically with an analyte. The N detection channels 10, 20, 30, 40 each have different detection sets or different combinations of detection sets. Each analyte out of a plurality of possible analyte is assigned a unique barcode representing readout signals 15, 25, 35, 45 produced for the N detection channels 10, 20, 30, 40. Each readout signal 15, 25, 35, 45 of the N readout signals 15, 25, 35, 45 is produced by a recognition molecule binding specifically to or reacting specifically with an analyte for each detection channel 10, 20, 30, 40 of the N detection channels 10, 20, 30, 40.

In an embodiment, the recognition molecules of the different sets are preloaded in the detection channels 10, 20, 30, 40 of the in vitro detecting device 1 and/or the kit, optionally with other reaction ingredients.

### Example

This example investigated how padlock probes could be used in three Eppendorf® tubes to detect presence of up to eight different analytes.
1. A sample comprising a nucleic acid molecule at a desired concentration was divided into three Eppendorf® tubes, such as 1 nM in 20 μl of volume in each Eppendorf® tube.
2. 10 nM of each padlock probe and SplintR® ligase and ligation buffer were mixed according to manufacturer instructions. 5 μl of the ligation mix was added to the sample in each Eppendorf® tube and incubated in 5 minutes at room temperature.
3. 0.5 μl (1U/μl) Exonuclease I (ExoI) and Lambda exonuclease was added to each Eppendorf® tube and incubated for 1 hour at 37° C. The enzymes were inactivated by heating to 95° C. for 5 minutes.
4. Ingredients for RCA were prepared by adding detection beads and Phi29 polymerase to an RCA mix according to manufacturer instructions. 15 μl RCA mixture was added to the ligation reaction product in each Eppendorf® tube and incubated for 25 minutes in 37° C.
   a. RCA mix-mix of bovine serum albumin (BSA), Phi29 buffer, dNTPs
   b. Detection beads-streptavidin-coated, colored, polystyrene beads, pre-incubated with biotinylate detection oligos of 20 nt

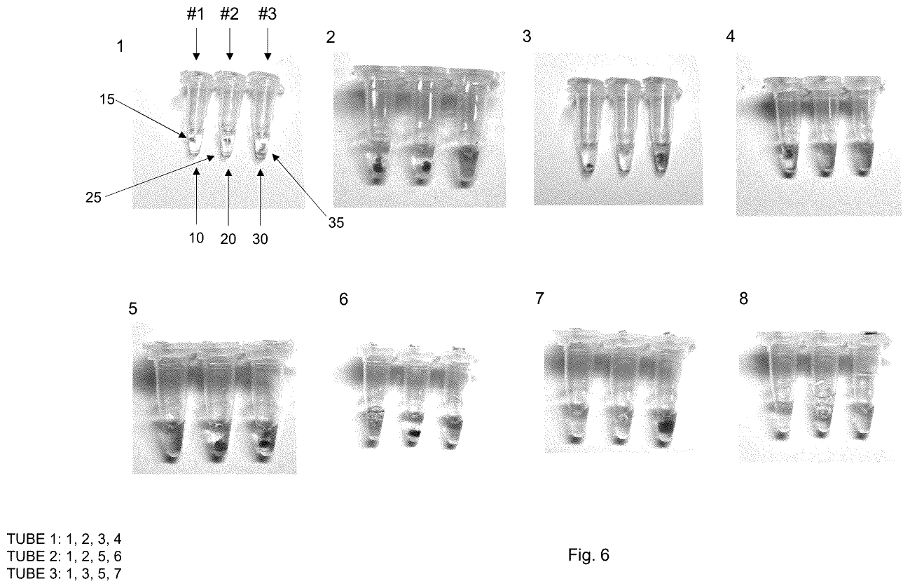

FIG. 6 illustrates the result of this example for eight different nucleic acid molecules as analytes and eight different padlock probes binding specifically to a respective analyte.

Padlock probes 1-4 were added to Eppendorf® tube no. 1, padlock probes 1, 2, 5, 6 were added to Eppendorf® tube no. 2 and padlock probes 1, 3, 5, 7 were added to Eppendorf® tube no. 3. The different figures indicate the result when the sample comprised analytes 1-8. For instance, in the upper figure to the left, a positive blue signal 15, 25, 35 was obtained in all Eppendorf® tubes indicating that the sample contained analyte no. 1. Correspondingly, the next upper figure numbered 2 shows the result of a sample comprising analyte no. 2 and produced a barcode with positive blue signal in Eppendorf® tubes 1, 2 but not 3.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-29. (canceled)

30. An in vitro detection method comprising:
contacting, for each detection channel of N≥2 detection channels, a sample with a plurality of recognition molecules, wherein
each recognition molecule of the plurality of recognition molecules binds specifically to or reacts specifically with a respective subset of at least one analyte;
the plurality of recognition molecules are divided into multiple detection sets comprising multiple recognition molecules of the plurality of recognition molecules;
the multiple recognition molecules of each detection set of the multiple detection sets produce a same readout signal when binding specifically to or reacting specifically with an analyte; and
the N detection channels each have different detection sets or different combinations of detection sets;
detecting, for each detection channel of the N detection channels, any readout signal produced by a recognition molecule binding specifically to or reacting specifically with an analyte; and
identifying an analyte in the sample based on a barcode representing the readout signals produced for the N detection channels.

31. The in vitro detection method according to claim 30, further comprising separating, for each detection channel of the N detection channels, any recognition molecule bound specifically to or reacted specifically with an analyte from recognition molecules not bound specifically to or reacted specifically with any analyte.

32. The in vitro detection method according to claim 31, wherein
contacting the sample comprises adding the sample to N lateral flow channels preloaded with the plurality of recognition molecules; and
separating any recognition molecule comprises separating, for each lateral flow channel of the N lateral flow channels, any recognition molecule bound specifically to or reacted specifically with an analyte from recognition molecules not bound specifically to or reacted specifically with any analyte.

33. The in vitro detection method according to claim 31, wherein separating any recognition molecule comprises separating, for each detection channel of the N detection channels, any recognition molecule bound specifically to or reacted specifically with an analyte from recognition molecules not bound specifically to or reacted specifically with any analyte by at least one of size-based filtration, centrifugation and affinity binding to a solid substrate.

34. The in vitro detection method according to claim 30, wherein
contacting the sample comprises adding the sample to N lateral flow channels preloaded with a plurality of recognition molecules comprising a respective detectable moiety and a plurality of recognition molecules comprising an epitope tag, wherein
each lateral flow channel of the N lateral flow channels comprises affinity molecules attached to a solid support and binding specifically to the epitope tag; and
a pair of a recognition molecule comprising a respective detectable moiety and a recognition molecule comprising the epitope tag binds specifically to a respective subset of at least one analyte.

35. The in vitro detection method according to claim 30, wherein each recognition molecule of the plurality of recognition molecules binds specifically to or reacts specifically with a respective subset of at least one analyte that is different than the respective subset of at least one analyte, to which other recognition molecules of the plurality of recognition molecules binds specifically or with which other recognition molecules of the plurality of recognition molecules reacts specifically.

36. The in vitro detection method according to claim 30, wherein
the N detection channels each have different combinations of recognition molecules in different detection sets; and
for each detection channel of the N detection channels, the multiple recognition molecules of each detection set of the multiple detection sets produce a readout signal that is different than readout signal(s) produced by recognition molecules of other detection set(s) of the multiple detection sets.

37. The in vitro detection method according to claim 30, wherein
the N detection channels each have different combinations of detection sets; and
the detection sets of each detection channel of the N detection channels comprise different combinations of recognition molecules of the plurality of recognition molecules.

38. The in vitro detection method according to claim 30, wherein the N detection channels each have different detection sets comprising different combinations of recognition molecules of the plurality of recognition molecules.

39. The in vitro detection method according to claim 30, wherein each recognition molecule of the plurality of recognition molecules binds specifically to or reacts specifically with a respective analyte.

40. The in vitro detection method according to claim 30, wherein the number of different recognition molecules, with which the sample is contacted, is larger than N.

41. The in vitro detection method according to claim 30, wherein each analyte has a unique barcode.

42. The in vitro detection method according to claim 30, wherein contacting the sample comprise contacting, for each detection channel number n of the N detection channels, the sample with Mₙ recognition molecules, wherein n=1 . . . N and each Mₙ≥2.

43. The in vitro detection method according to claim 42, wherein Mₙ>N.

44. The in vitro detection method according to claim 30, wherein
the plurality of recognition molecules are divided into Cₙ≥2 detection sets for detection channel number n of the N detection channels, wherein n=1 . . . N; and
identifying the analyte comprises identifying the analyte in the sample out of up to
$${\prod }_{n = 1}^{N} {C}_{n} or {\prod }_{n = 1}^{N} ( {C}_{n} + 1 ) - 1$$
signals produced for the N detection channels.

45. The in vitro detection method according to claim 44, wherein
the plurality of recognition molecules are divided into C≥2 detection sets; and
identifying the analyte comprises identifying the analyte in the sample out of up to C^{N} or (C+1)^{N}−1 possible analytes based on the barcode representing the readout signals produced for the N detection channels.

46. The in vitro detection method according to claim 30, wherein
the plurality of recognition molecules are divided into Cₙ≥2 detection sets for detection channel number n of the N detection channels, wherein n=1 . . . N; and
contacting the sample comprise contacting, for each detection channel number n of the N detection channels, the sample with a plurality of recognition molecules out of up to
$${\prod }_{n = 1}^{N} {C}_{n} or {\prod }_{n = 1}^{N} ( {C}_{n} + 1 ) - 1$$
recognition molecules.

47. The in vitro detection method according to claim 46, wherein
contacting the sample comprise contacting, for each detection channel number n of the N detection channels, the sample with
$${\prod }_{n = 1}^{N} {C}_{n} or {\prod }_{n = 1}^{N} ( {C}_{n} + 1 ) - 1$$
recognition molecules;
detecting the readout signal comprises detecting, for each detection channel number n of the N detection channels, a readout signal cₙ produced by a recognition molecule binding specifically to or reacting specifically with an analyte, wherein each cₙ represents a readout out of Cₙ possible readouts; and
identifying the analyte comprises identifying the analyte in the sample based on the barcode [c₁, . . . , c_{N}].

48. The in vitro detection method according to claim 46, wherein
the plurality of recognition molecules are divided into C≥2 detection sets; and
contacting the sample comprise contacting, for each detection channel of the N detection channels, the sample with a plurality of recognition molecules out of up to C^{N} or (C+1)^{N}−1 recognition molecules.

49. The in vitro detection method according to claim 48, wherein
contacting the sample comprise contacting, for each detection channel number n of the N detection channels, the sample with C^{N} or (C+1)^{N}−1 recognition molecules;
detecting the readout signal comprises detecting, for each detection channel number n of the N detection channels, a readout signal cₙ produced by a recognition molecule binding specifically to or reacting specifically with an analyte, wherein each cₙ represents a readout out of C possible readouts; and
identifying the analyte comprises identifying the analyte in the sample based on the barcode [c₁, . . . , c_{N}].

50. The in vitro detection method according to claim 30, wherein
the N detection channels each have different combinations of detection sets; and
for each detection channel of the N detection channels, the multiple recognition molecules of each detection set of the multiple detection sets produce a color signal that is different than color signal(s) produced by recognition molecules of other detection set(s) of the multiple detection sets;
detecting the readout signal comprises detecting, for each detection channel of the N detection channels, a color signal produced by the recognition molecule binding specifically to or reacting specifically with the analyte; and
identifying the analyte comprises identifying the analyte in the sample based on a barcode representing the color signals produced for the N detection channels.

51. The in vitro detection method according to claim 30, wherein
detecting the readout signal comprises detecting, for each detection channel of the N detection channels, a binary readout signal produced by the recognition molecule binding specifically to or reacting specifically with the analyte; and
identifying the analyte comprises identifying the analyte in the sample based on a barcode representing the binary signals produced for the N detection channels.

52. The in vitro detection method according to according to claim 30, wherein
detecting the readout signal comprises detecting, for each detection channel of the N detection channels, a readout signal representing a spatial location of a readout in the detection channel and produced by the recognition molecule binding specifically to or reacting specifically with the analyte; and
identifying the analyte comprises identifying the analyte in the sample based on a barcode representing the spatial locations of the readouts produced for the N detection channels.

53. The in vitro detection method according to claim 30, wherein
the analytes are different proteins; and
the recognition molecules are selected from the group consisting of aptamers, antibodies, and antigen-binding fragments thereof.

54. The in vitro detection method according to claim 30, wherein
the analytes are different nucleic acid molecules, and
the recognition molecules are nucleic acid probes.

55. The in vitro detection method according to claim 30, wherein
a predefined number M<N of the readout signals representing the barcode have a respective predefined readout; and
identifying the analyte comprises identifying a mixture of at least two different analytes in the sample based on a barcode with more than M readout signals having the respective predefined readout.

56. The in vitro detection method according to claim 30, wherein
the multiple recognition molecules of each detection set of the multiple detection sets produce a predefined color signal that is different than predefined color signal(s) produced by recognition molecules of other detection set(s) of the multiple detection sets;
detecting the readout signal comprises detecting, for each detection channel of the N detection channels, a color signal produced by the recognition molecule binding specifically to or reacting specifically with the analyte; and
identifying the analyte comprises identifying a mixture of at least two different analytes in the sample if the color signal produced for at least one detection channel is different than any of the predefined color signals.

57. An in vitro detection device comprising:
N≥2 detection channels; and
a plurality of recognition molecules, wherein
each recognition molecule of the plurality of recognition molecules binds specifically to or reacts specifically with a respective subset of at least one analyte;
the plurality of recognition molecules are divided into multiple detection sets comprising multiple recognition molecules of the plurality of recognition molecules;
the multiple recognition molecules of each detection set of the multiple detection sets produce a same readout signal when binding specifically to or reacting specifically with an analyte; and
the N detection channels each have different detection sets or different combinations of detection sets, wherein
each analyte out of a plurality of possible analyte is assigned a unique barcode representing readout signals produced for the N detection channels; and
each readout signal of the N readout signals is produced by a recognition molecule binding specifically to or reacting specifically with an analyte for each detection channel of the N detection channels.

58. The in vitro detection device according to claim 57, further comprising a detector configured to detect, for each detection channel of the N detection channels, a readout signal produced by a recognition molecule binding specifically to or reacting specifically with an analyte.

59. The in vitro detection device according to claim 58, further comprising a processor configured to process the readout signals and determine an analyte identifier identifying an analyte out of the plurality of analytes based on a barcode representing the readout signals produced for the N detection channels.
